(19) United States
(12) Patent Application Publication
(10) Pub. No.: US 2015/0335871 A1
(43) Pub. Date: Nov. 26, 2015

(54) METALLIC MICRONEEDLES

(71) Applicant: Chee Yen LIM, (US)

(72) Inventor: Chee Yen LIM, Singapore (SG)

(21) Appl. No.: 14/652,781

(22) PCT Filed: Jan. 7, 2013
(86) PCT No.: PCT/SG2013/000007

§ 371 Date: Jun. 16, 2015

(51) Int. Cl. A61M 37/00

(52) U.S. Cl. CPC A61M 37/0015; A61M 2037/0053; A61M 2037/0046

(57) ABSTRACT

The present invention relates to fabrication of metallic microneedles 200 which involves two process steps: firstly providing a rough cut on a metallic block to form approximate microneedle shapes, and secondly providing a finishing cut on the rough-cut metallic block to achieve final microneedles with required surface smoothness and tip sharpness. Providing the rough cut can be achieved by using conventional manufacturing methods such as Electro-Discharge Machining (EDM) or Computerised Numerical Controlled (CNC) machining by first forming a rough cut a metallic block 500 of the microneedle array 600. The finishing cut is achieved by polishing the individual microneedles 720 on the rough-cut metallic block 500 to desired sharpness and surface finish. The microneedles 720 formed are partial or full pyramidal structures which normally have heights of 100 microns to 1,000 microns with sharp tips that can penetrate the skin painlessly and effectively. Hollow microneedles can be achieved by drilling a through hole on the solid microneedles.

### FIELD OF THE INVENTION

The present invention relates to fabricating microneedles for transdermal and intradermal drug delivery, particularly, it relates to direct machining of solid and hollow microneedles via conventional machining methods such as CNC machining, wire electro-discharge machining, profile grinding, etc.

### BACKGROUND OF THE INVENTION

Drug is normally administered or brought to contact with a body via topical, enteral (oral) and parenteral (injection) means. In topical administration, the applied drug is supposed to take effect locally, while in enteral and parenteral administration, the drug effect is systemic (entire body). Transdermal drug delivery is a relatively new form of drug administration targeting a systemic delivery by making drugs available on the skin painlessly. This is different than topical method which is intended to target local delivery. This is the first obstacle as the efficacy of the drug is not guaranteed even the drug can be successfully delivered to the body. Solid microneedles are normally chosen for transdermal drug delivery because potent medicinal substances can be coated on the microneedles in dry form and be delivered to the skin by inserting the solid microneedles into the skin. Intradermal drug delivery is injecting medicinal liquid into epidermal layer of skin. It normally involves vaccines as skin contains plenty of antigen-presenting cells. There are only a small number of vaccines that are delivered intradermally, they are namely BCG (against tuberculosis) and anti-rabies vaccines due to a complex procedure that requires skill. One clear advantage of delivering vaccines transdermally or intradermally is that the required volume of the vaccines is significantly less than that required by intramuscular injections.

The second obstacle in transdermal or intradermal delivery is to overcome the outermost layer of the skin, called stratum corneum, which is made up by dead cells that are pushed to the outermost of the body. Stratum corneum forms a formidable layer (20 microns on average) to isolate and protect the body. Because of this formidable layer, only a few small-molecule drugs can be administered via transdermal route. Over two decades, transdermal delivery is complemented by microneedles to overcome stratum corneum to allow faster delivery rate and larger-molecule drugs to be delivered. Since microneedles physically breach or perforate the skin to make way for the drug, the effectiveness is excellent and consistent.

After examining the first two obstacles, there is the last obstacle that is in the way of transdermal delivery, i.e. the cost of microneedles, which includes the initial capital investment and subsequent operational expenditure. Most microneedles developed in the lab lack the capability to scale up with acceptable cost efficiency. The Microneedle technology for transdermal drug delivery has been around for two decades and there is yet any commercial product on the market to date. One major hindrance for commercialization is the production cost and mass manufacturability, which the present invention seeks to address.

There are plenty of microneedles made by various methods in the literature. There are mainly two broad categories, namely direct fabrication approach and moulding approach. In the direct fabrication approach, materials are usually removed from a work piece, e.g. a metal sheet, a silicon wafer and so on to form the microneedles, whereas in the moulding approach, a mould is first constructed which is followed by forming the microneedles taking the shapes of the mould. The direct fabrication approach directly produces metallic or silicon microneedles, from metal sheet with chemical or photo-etching as seen in patent issued to Alza Corp. (U.S. Pat. No. 6,219,574), or from silicon wafer with dry or wet etching process as seen in patents issued to Nanopass Ltd (U.S. Pat. No. 6,533,949). These fabrication methods are not the conventional methods for mass production and the production costs are too high for making disposable microneedles. On the other hand, the moulding approach normally involves forming plastic materials into plastic microneedles, as seen in patents issued to Procter & Gamble (U.S. Pat. No. 6,471,903) and to 3M (U.S. Pat. No. 8,088,321). Since plastic materials are much lower in strength and hardness compared to metals such as stainless steel, moulded plastic microneedles tend to bend or break leaving tiny fractions in the skin after use. Hence, metallic microneedles are strongly preferred over plastic microneedles for this reason. Although moulding approach includes electro-forming of metallic microneedles on a mould, we think it is not a viable manufacturing approach because the electro-forming process takes very long time and involves highly toxic and carcinogenic chemicals.

Only patent '574 issued to Alza Corp. involves fabrication of metallic microneedles. It is learned from the patent that the microneedles are micro-blades that are formed in-plane on a titanium sheet or stainless steel sheet, and the micro-blades are bent 90 degrees perpendicular to the sheet to form protruding micro-blades. The forming of the in-plane micro-blades involves masking the sheet and etching the sheet which involves toxic chemicals. The subsequent bending process, which involves punch and die to push each micro-blade 90 degrees out of plane is technically challenging. The last but not least, the micro-blades sharpness and edges are determined by the thickness of the sheet, as there is no sharpening process to further sharpen the tips and cutting edges. This leaves the tips and edges to be between 50 microns to 100 microns because this is the thinnest sheets (or foils) available with considerable strength. In conclusion, these micro-blades may be hard and expensive to make and too blunt to penetrate skin.

As such, there exists a long-felt need for simple and efficient mass-producible microneedles that are sharp and strong enough for effective skin penetration and drug delivery. The present invention aims to provide a solution to this long-felt need.

### SUMMARY OF THE INVENTION

The present invention relates to fabricating metallic microneedles using conventional manufacturing methods such as CNC machining, precision electro-discharge-machining, profile grinding and other widely used techniques by first providing a rough cut of the microneedle array and followed by polishing the microneedles to required sharpness and surface finish.

In the preferred embodiment, a metallic block which is substantially planar is machined into long sharp ridges of 300 microns to 700 microns height which are parallel to each other. The parallel ridges are formed through machining on one of the substantial planar surface of the metallic block. The material of the metallic block can be chosen from any bio-compatible metals, including stainless steel, titanium and any other suitable materials. Subsequently, the metallic block is rotated 90 degrees and the similar machining patterns are applied to the same substantially planar surface of the block. In this manner, an array of pyramids with height 300-700 microns are fabricated. Apparently, the height and the width of the ridges are respectively the height and the width of the pyramids. These pyramids are microneedles which is able to penetrate the skin painlessly and effectively.

### BRIEF DESCRIPTION OF THE DRAWINGS

FIG. 1 is a perspective view of metallic block 10 comprising clusters of microneedles 30.

FIG. 2 is a close-up view of one microneedle array 100 separated from the metallic block 10.

FIG. 3 is a perspective view of a microneedle array 100 assembled with adhesive patch 240 and a backing layer 220.

FIG. 4 is an exploded view of the assembled microneedle array 200 in FIG. 3.

FIG. 5 is a perspective view of a packed microneedle array 300 which comprises an assembled microneedle array 200 in FIG. 3 and a lid 320.

FIG. 6 is a schematic diagram showing an ideal skin penetration depth of a microneedle array 200 achieving painless and effective drug delivery.

FIG. 7 shows a schematic diagram on the process flow of attaining microneedles, which comprises providing a metal block 600, applying conventional machining methods to provide a rough cut of microneedles on the metal block 600, and finally performing finishing cut by polishing the rough cut microneedles 620 to form finished cut microneedles 720.

FIG. 8 shows two examples of rough cuts, one comprising tapered pillars 700 and one comprising straight pillars 800.

FIG. 9 shows an example of forming a rough cut 1100 by electro-discharge machining method, in which a pre-cut copper electrode containing a negative pattern of straight pillars is used to spark off material from the rough cut block 1100.

FIG. 10 shows a perspective view of a finishing cut process in which a grinding wheel 1200 turns in one direction 1220, and moves linearly across the rough cut block 1400, producing finished cut block 1400.

FIG. 11 is a close-up view of FIG. 10 where the turning grinding wheel 1220 cuts the rough cut block 1400 in the traces provided in the figure.

FIG. 12 is a magnified view of a mouse skin cut and flipped out to confirm effective penetration and delivery of dye into the skin without damaging or puncturing the cartilage under the skin.

FIG. 13 shows a few microneedle patches are stuck on a user's finger tips to generate micro-holes particularly for cosmetic applications.

### DETAILED DESCRIPTION OF THE INVENTION

For the purpose of illustrating the principles of the present invention, reference will now be drawn to the embodiments illustrated herein and specific language will be used to describe the same. It should be understood that no limitation of the scope of the present invention by these embodiments and language is intended. Any alterations and further modifications and applications of the principles of the present invention by a person skilled in the art shall fall in the scope of the present invention.

### Design and Packaging of Microneedle Arrays

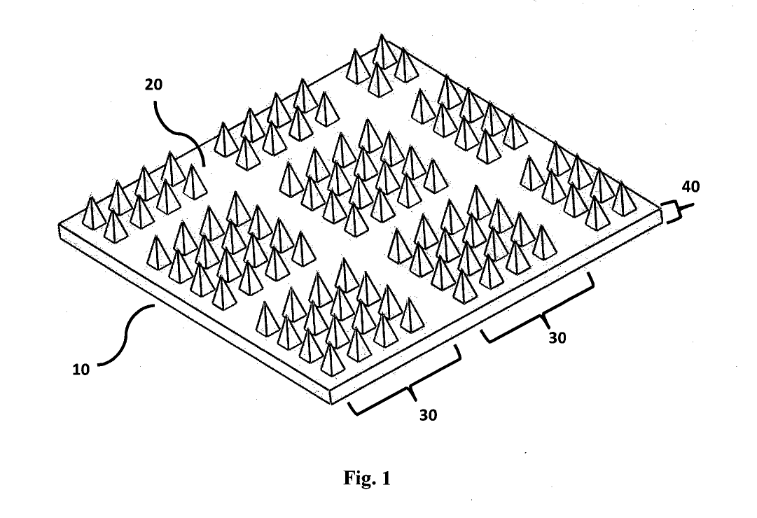

FIG. 1 shows a substantially planar metallic block 10 having a work plane 20 on which clusters of microneedle arrays 30 stand. The work plane 20 may have 100 mm×100 mm or larger surface area for machining. The thickness of the base 40 is the remaining height after the clusters of microneedles 30 have been machined, which typically has a thickness of less than 1 mm. More specifically, the thickness of the base 40 has a measurement of less than 0.5 mm or between 0.2 mm to 0.4 mm. The footprint of the microneedle clusters 30 can be varied to increase the number of microneedles per cluster, but this will reduce the number of clusters per block 10.

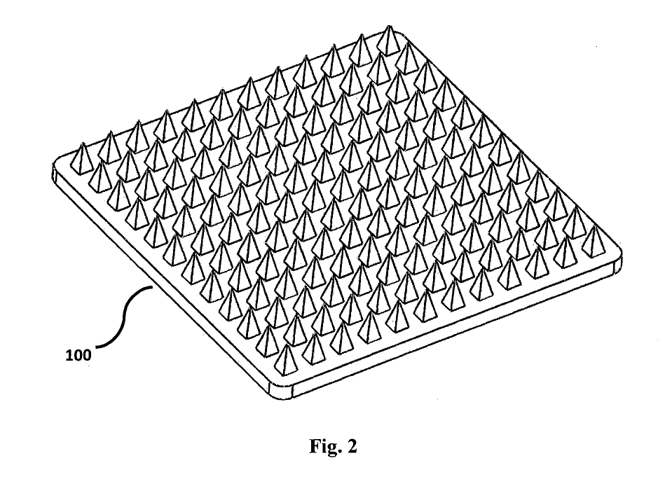

FIG. 2 shows a microneedle array 100 that contains one of the microneedle clusters 30 which is separated from the metallic block 10. Each microneedle is made of a 300-micron tall pyramid with a sharp tip, i.e. with a tip size of less than 10 microns, and more specifically, with a tip size of less than 1 micron, as well as a tiny base area of 150 microns×150 microns to 200 microns x 200 microns. It is well known in the art that the barrier to be breached by the microneedles is stratum corneum, which is on average 20 microns in thickness. However, microneedles are made much longer due to flexibility and stretch-ability of the skin, the typical length is in the range of 200 microns to 500 microns, or even longer.

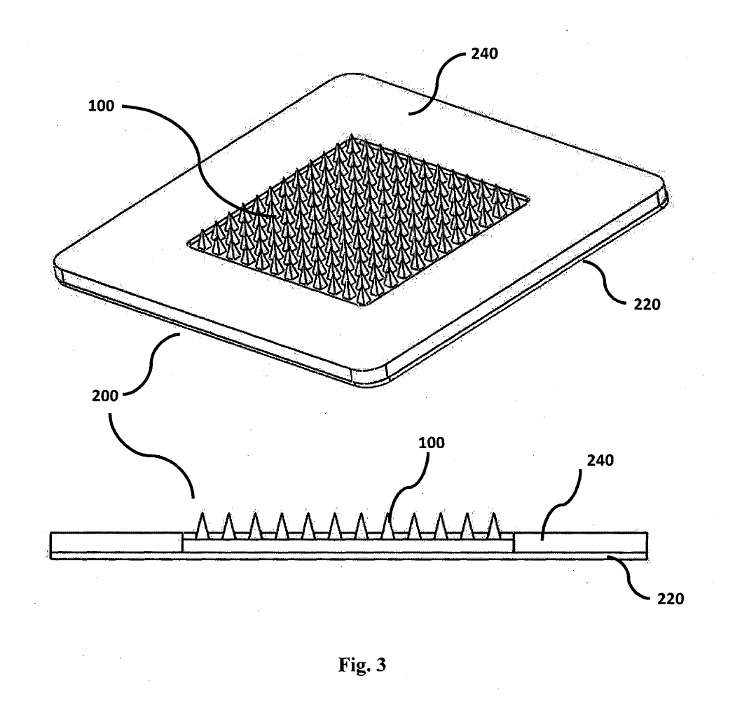

FIG. 3 shows a microneedle array 100 assembled with a backing layer 220 and an adhesive patch 240. FIG. 3 is a typical assembled microneedle array 200 currently widely used for transdermal drug delivery applications. A drug or pharmaceutically active ingredient is coated on the individual microneedles in the assembled microneedle array 200. The backing layer 220 serves as a connector holding the microneedle array 100 and the adhesive patch 240 together. The adhesive patch 240 serves the essential function of holding the microneedles into the skin for as long as required till the drug delivery is complete.

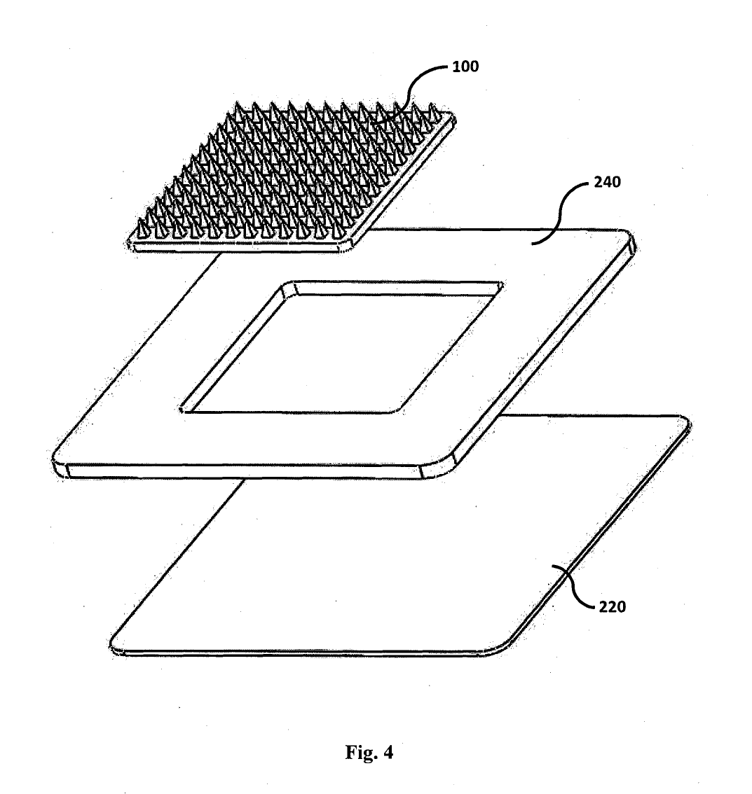

FIG. 4 shows an exploded view of the assembled microneedle array 200 in FIG. 3.

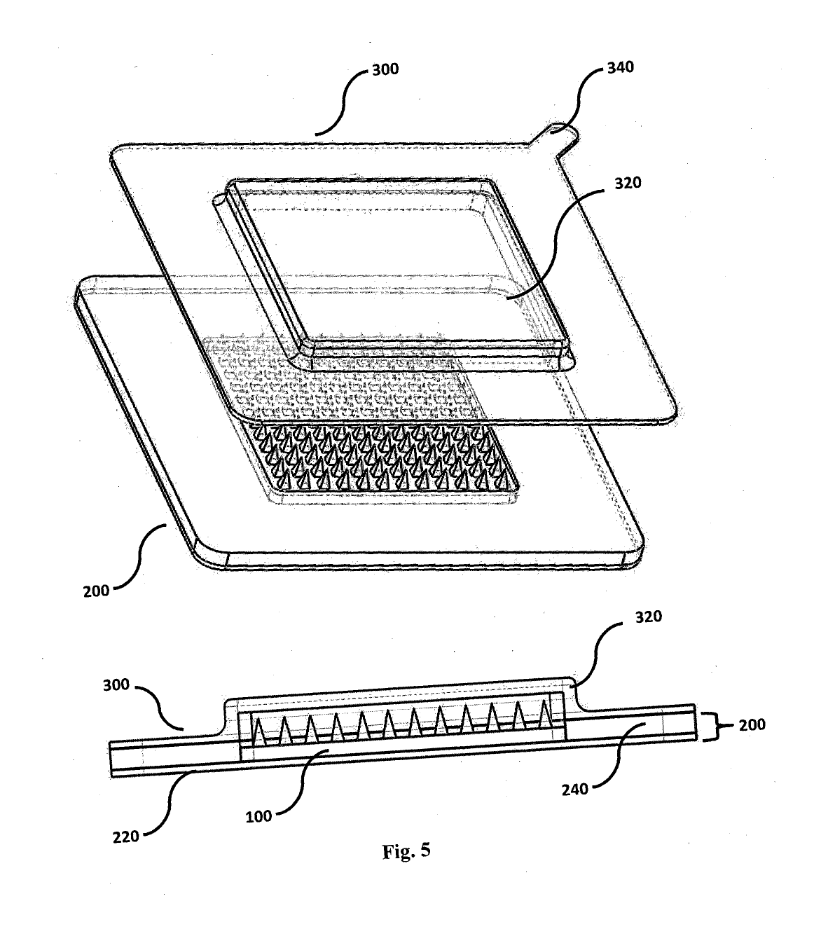

FIG. 5 shows a typical packaging of the assembled microneedle array 200. The packed microneedle array comprises the assembled microneedle array 200 and a lid 300 which further comprises a protrusion centre 320 to cater to the microneedle array 200 and a tearing tab 340 for easy removal of the lid 300.

### Application of Microneedle Arrays

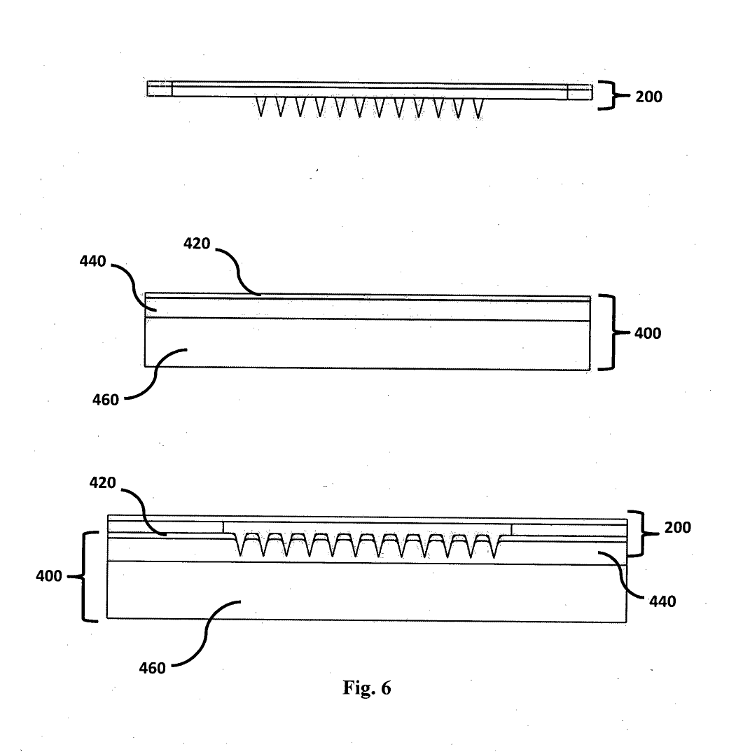

FIG. 6 shows an assembled microneedle array 200 and a typical mammal skin profile 400, comprising the outermost layer stratum corneum 420, epidermal layer 440, and dermal layer 460. For effective and painless drug delivery, the microneedles have to penetrate the stratum corneum 420 and stop at the epidermal layer 440. Failing to penetrate stratum corneum 420 will make the delivery attempt unsuccessful and failure to penetrate within the epidermal layer 440 will induce trauma (pain). The second figure in FIG. 6 shows the desired penetration outcome in which the microneedles on the assembled microneedle array 200 sufficiently penetrate stratum corneum 420 and stay within the epidermal layer 440, without reaching the dermal layer 460.

### Manufacturing of Microneedle Arrays

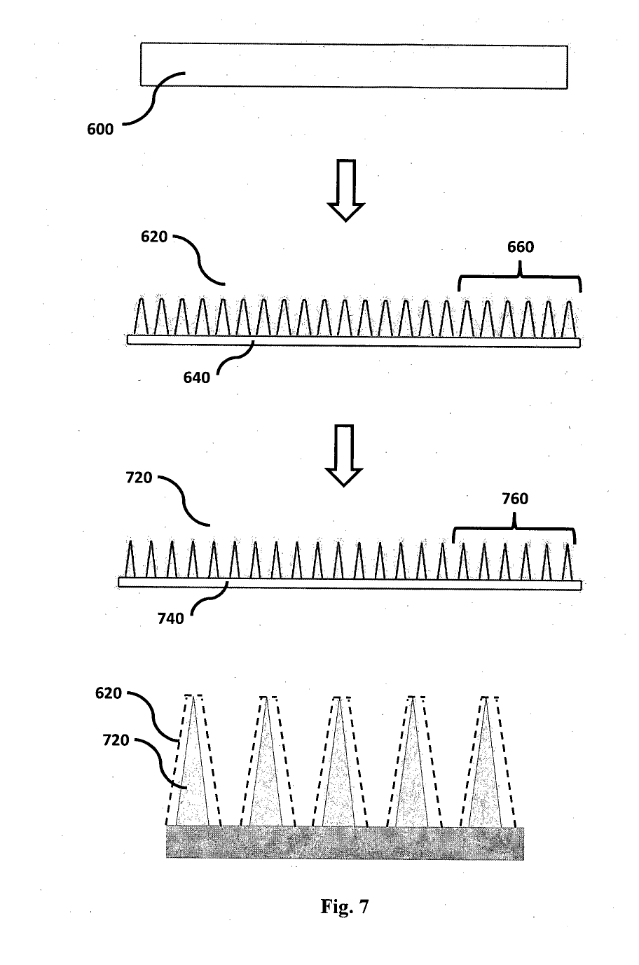

FIG. 7 provides a process flow for the manufacturing of microneedle arrays, essentially comprising providing a metal block 600, implementing a rough cut 620 on the metal block 600, and finally performing a finishing cut 720 on the rough cut 620. The rough cut 620 is swiftly accomplished by conventional machining methods including grinding, EDM (electro-discharge machining), metal injection moulding, and so on. The rough cut 620 has a substrate 640 which acts as a mattress for all rough cuts of microneedles to stand on, and the rough cuts 620 can be in clusters of microneedle rough cuts 660. The last figure of FIG. 7 shows the dimensional difference of the rough cuts 620 and the finished cuts (microneedles) 720. The rough cuts 620 are less sharp and generally slightly bigger than the finished cuts 720. The finished cuts 720 are often done by profile grinding so that the tips can be less than 10 um, or more particularly less than 1 um. The finished cuts 720 stands on a substrate 740 and the finished microneedles can form clusters of finished microneedles 760. It is highly preferable that the finished products are subjected to electro-polishing to further sharpen the tips and to remove burrs generated during the grinding process.

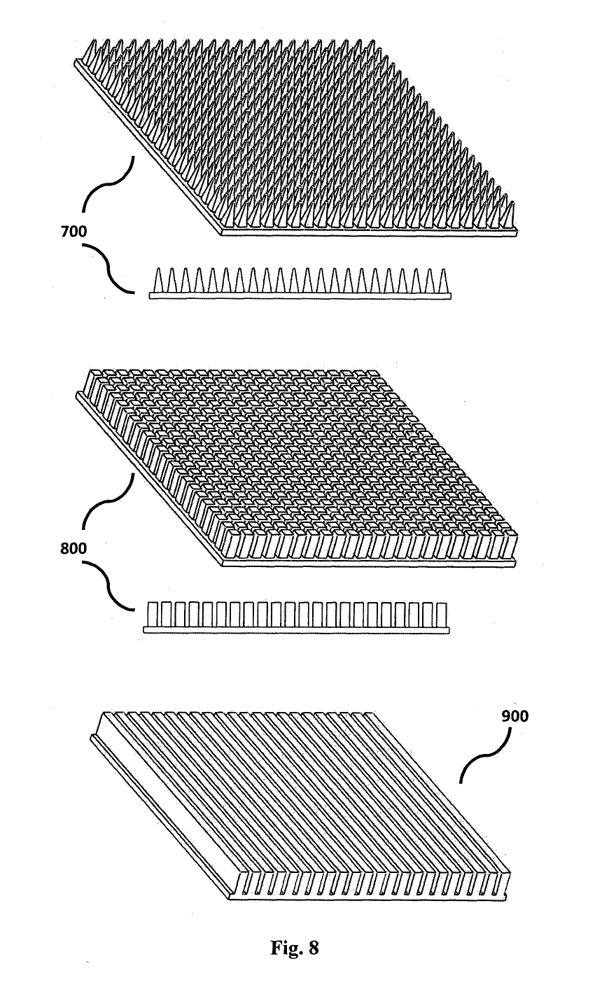

FIG. 8 shows some examples of rough cuts 700, 800, and 900. Rough cut 700 features trapezium pillars which can be turned into sharp microneedles in the finishing cut process. Rough cut 800 features straight pillars which can also be polished into sharp microneedles via profile grinding. Rough cut 900 features ridges that can be turn into pillars which subsequently can be polished into finished microneedles. Rough cut 900 may be an intermediate step of forming a rough cut of 700 or 800.

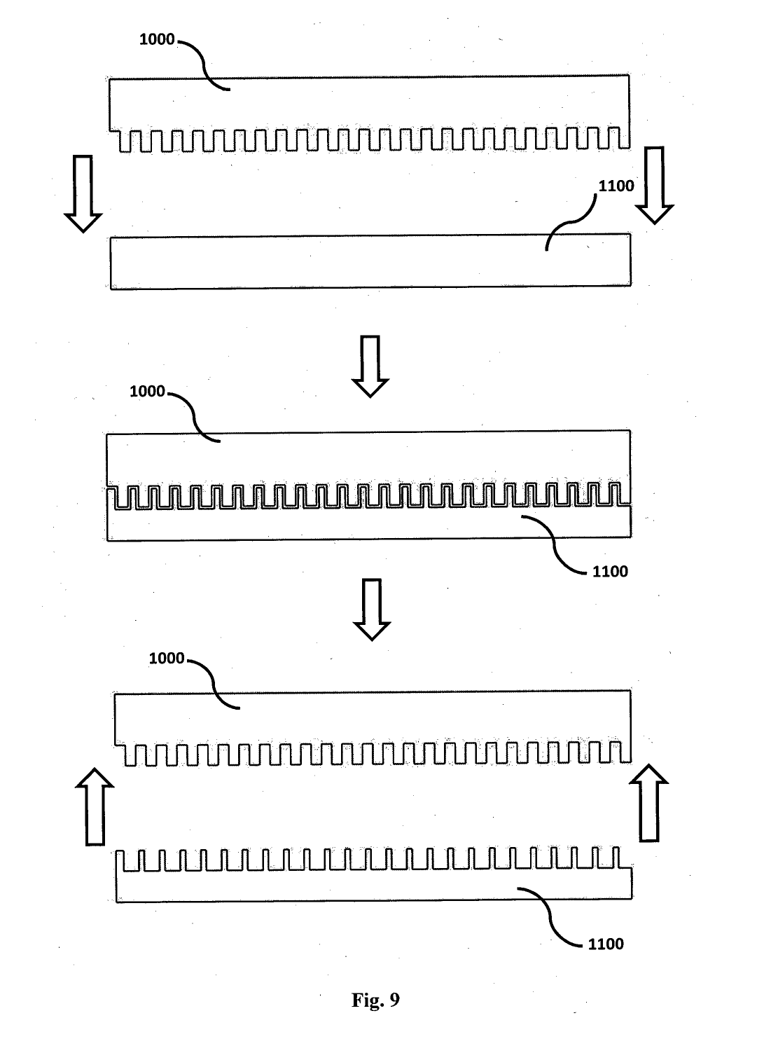

FIG. 9 shows an example of producing a rough cut by EDM (electro-discharge machining). A copper electrode 1000 formed with a negative pattern of the rough cut is passed through with pulsed current and put in close proximity to a metallic block 1100. The required time to produce a typical rough cut depends on the amplitude of current, sparking removal rate and

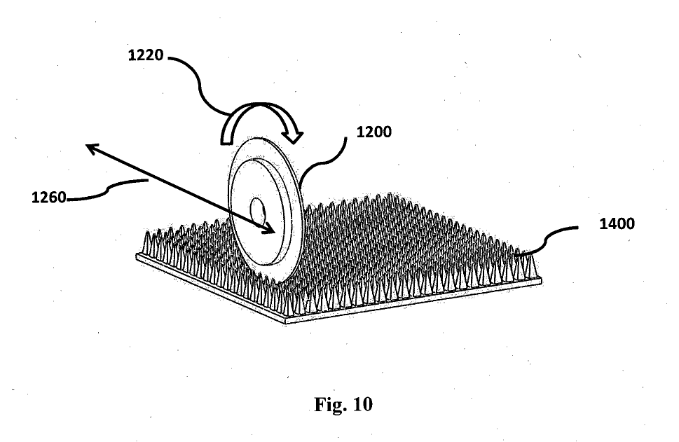

FIG. 10 shows the finishing cut process on a rough cut block 1400 by profile grinding using a grinding wheel 1200 which rotates around an axis (not shown) with a rotation direction 1220, cutting across the rough cut block 1400 linearly and reciprocally. The rotating grinding wheel polishes the rough cut microneedles one row at a time and different wheels may be used for opposite angles or opposite faces. The rough cut block 1400 may be turn 90 degrees for polishing the other pyramid surfaces till all surfaces are polished. It can be thought of easily of a person skilled in the art to use bi-angled grinding wheel to polish two surfaces at one time.

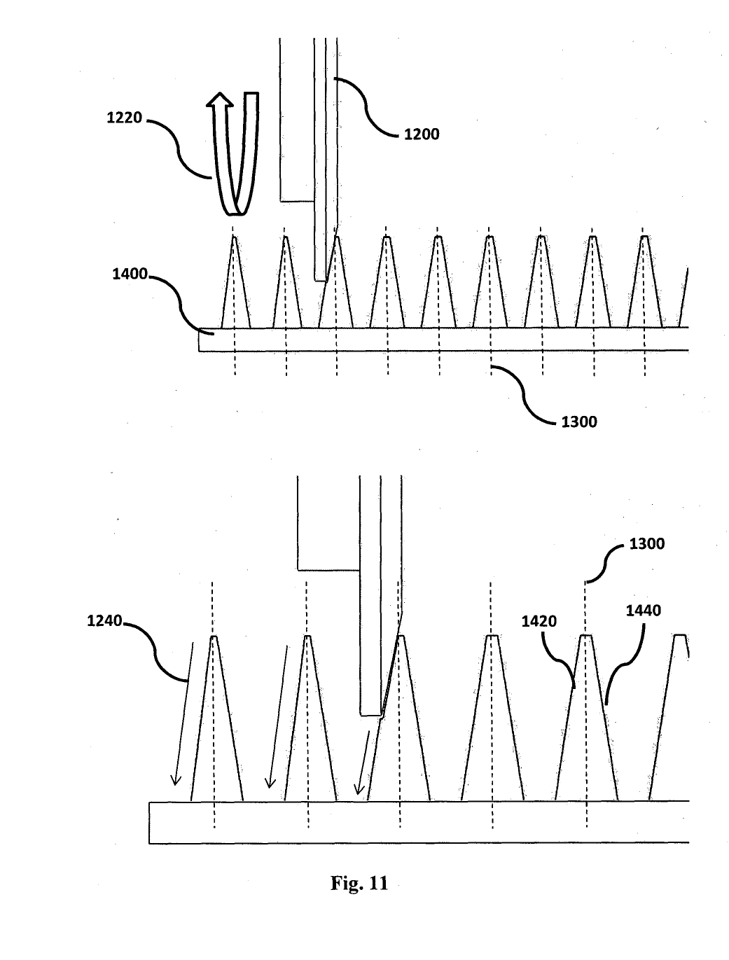

FIG. 11 is a close up schematic diagram of FIG. 10. The grinding wheel 1200 having a predetermined cutting angle moves down from above removing material from the rough cut block 1400, thereby polishing the surface and sharpening the tips of the pyramids (microneedles).

The last optional process is electro-polishing which removes burrs that build up during the finishing cut process by profile grinding. The building up of burrs may be due to the chips or debris that is cut from the microneedles is fused back on the surface due to extreme heat generated during the grinding process. The electro-polishing process generally removes (oxidizes) metal but the surface irregularities induce high electric field that accelerates the material removal. Normally electro-polishing can be controlled to remove 1-5 microns for achieving de-burring effect, and can even be used to sharpen the tips of the microneedles by deliberately extending the electro-polishing duration. A typical set of parameters for effectively de-burring the microneedles is 8 Volts for 20 seconds. The current varies with the effective area of electro-polishing.

Another optional process is drilling through holes in the microneedles to form hollow microneedles. This process is very straightforward from manufacturing point of view, except that whether to form the microneedles first then drill through holes within them or vice versa is process dependent. The persons skilled in the art should decide whether to drill the through holes first or later once he decides the method of drilling.

### Animal Testing using Microneedle Arrays

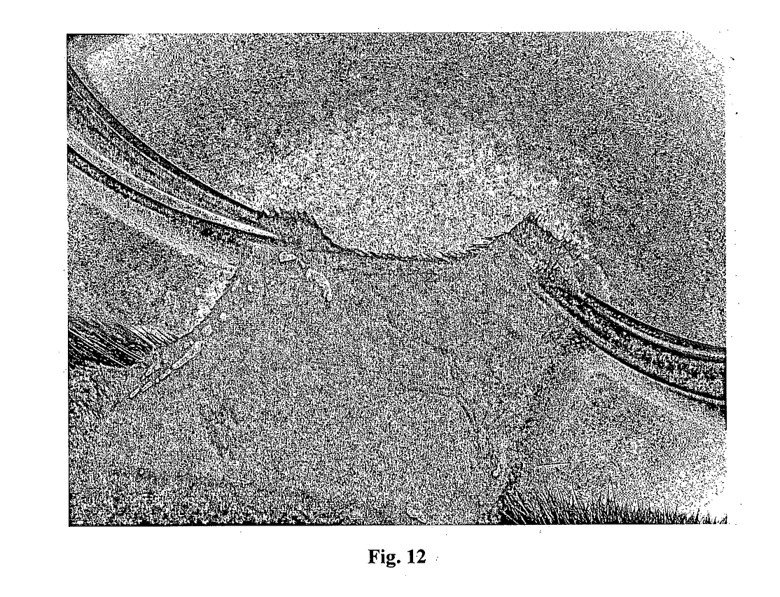

FIG. 12 shows a magnified view of a mouse skin cut and flipped out to confirm effective penetration and delivery of dye into the skin without damaging or puncturing the cartilage under the skin. A patch system from FIG. 5 was used to apply microneedle array of 0.5 mm height made by the methods described herein and to hold it in the skin for one minute. The stain was due to the blue dye pre-coated on the microneedles. This is a clear evidence of effective skin penetration by microneedle arrays.

### Examples of Microneedle Products

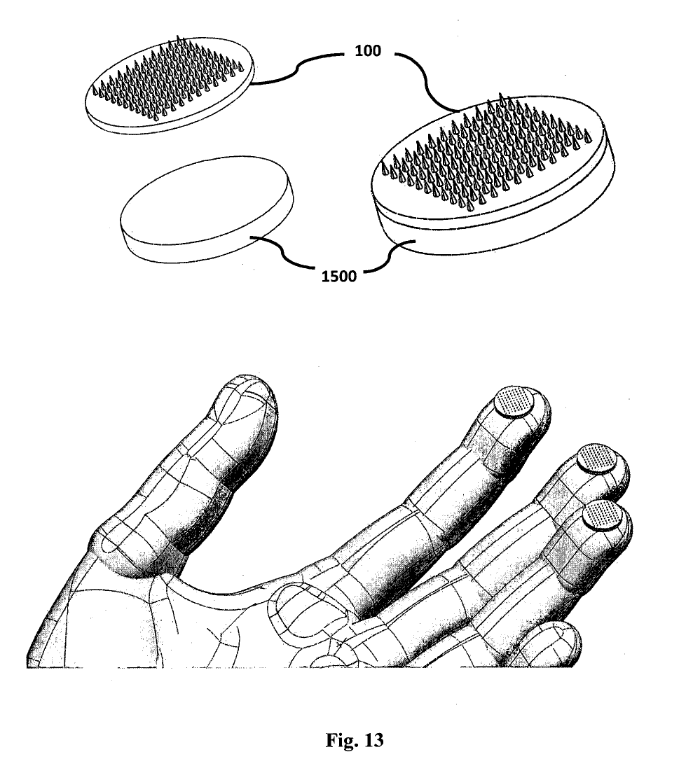

FIG. 13 shows a simple microneedle array product comprising a microneedle array 100 and a double-sided adhesive 1500. The double-sided adhesive 1500 holds the microneedle array 100 on a user's finger tips so that the user can use his fingers to sense and apply the microneedle arrays 100 to a desired skin spot, normally on his face.

We claim:

1. A method of fabricating metallic microneedles, comprising
a. Firstly providing a rough cut on a metallic block to form microneedle arrays with approximate shape;
b. Secondly providing a finishing cut on the metallic block to form microneedle arrays with final shape with required surface smoothness and tip sharpness;
wherein the finishing cut to produce smooth and sharp microneedles are achieved by removing material for the metallic block.

2. A method of fabricating metallic microneedles in claim 1, wherein the rough cut is achieved via electro-discharge machining methods.

3. A method of fabricating metallic microneedles in claim 1, wherein the rough cut is achieved via metal injection moulding method.

4. A method of fabricating metallic microneedles in claim 1, wherein the rough cut is achieved via grinding methods.

5. A method of fabricating metallic microneedles in claim 1, wherein the approximate shape of microneedle arrays in the rough cut has straight walls.

6. A method of fabricating metallic microneedles in claim 1, wherein the approximate shape of microneedle arrays in the rough cut has tapered walls.

7. A method of fabricating metallic microneedles in claim 1, wherein the finishing cut is achieved via profile grinding method.

8. A method of fabricating metallic microneedles, comprising
a. Firstly providing a rough cut on a metallic block to form microneedle arrays with approximate shape;
b. Secondly providing a finishing cut on the metallic block to form microneedle arrays with final shape with required surface smoothness and tip sharpness;
c. Thirdly providing a burr removal procedure
wherein the finishing cut to produce smooth and sharp microneedles are achieved by removing material for the metallic block.

9. A method of fabricating metallic microneedles in claim 8, wherein the rough cut is achieved via electro-discharge machining methods.

10. A method of fabricating metallic microneedles in claim 8, wherein the rough cut is achieved via metal injection moulding method.

11. A method of fabricating metallic microneedles in claim 8, wherein the rough cut is achieved via grinding methods.

12. A method of fabricating metallic microneedles in claim 8, wherein the approximate shape of microneedle arrays in the rough cut has straight walls.

13. A method of fabricating metallic microneedles in claim 8, wherein the approximate shape of microneedle arrays in the rough cut has tapered walls.

14. A method of fabricating metallic microneedles in claim 8, wherein the finishing cut is achieved via profile grinding method.

15. A method of fabricating metallic microneedles in claim 8, wherein the burr removal procedure is electro-polishing method.
